(19) United States
(12) Patent Application Publication
(10) Pub. No.: US 2007/0005043 A1
(43) Pub. Date: Jan. 4, 2007

(54) Osmotic Intraosseous Drug Delivery System

(75) Inventor: Rolfe C. Anderson, Saratoga, CA (US)

Correspondence Address: DEWIPAT INCORPORATED, P.O. BOX 1017, CYPRESS, TX, 77410-1017, US

(73) Assignee: ALZA CORPORATION, Mountain View, CA (US)

(21) Appl. No.: 11/428,232

(22) Filed: Jun. 30, 2006

Related U.S. Application Data
(60) Provisional application No. 60/695,621, filed on Jun. 30, 2005.

(51) Int. Cl. A61K 9/22; A61C 5/00

(52) U.S. Cl. 604/890.1; 433/217.1

(57) ABSTRACT

An osmotic intraosseous drug delivery device includes an artificial crown having a cavity defined therein and at least one inlet port in communication with the cavity. The device also includes an osmotic module including an osmotic agent and a drug formulation. The osmotic module is adapted to be inserted in the cavity in order to allow fluid received in the cavity through the inlet port to activate the osmotic module. The osmotic module has an outlet port through which the drug formulation can be dispensed.

### CROSS-REFERENCE TO RELATED APPLICATIONS

This application claims priority from U.S. provisional application 60/695621 filed Jun. 30, 2005, the content of which is incorporated herein in its entirety by reference.

### BACKGROUND OF INVENTION

The invention relates to continuous or patterned infusion of therapeutic agents.

Continuous or patterned infusion of therapeutic agents such as used in treatment of conditions like diabetes, Parkinson's disease, Hepatitis C, epilepsy, hypertension, congestive heart failure (CHF), muscular sclerosis (MS), and chronic pain can result in improved efficacy and reduced side effects. Parenteral infusion has been provided by external infusion pumps or pump implants. With external infusion pumps, the user must maintain the infusion site and deal with possible infection. Implants are more invasive and are not accessible for maintenance and control, although some may be refilled by injection.

From the foregoing, there continues to be a desire for an improved method of providing continuous or patterned infusion of therapeutic agents.

### SUMMARY OF INVENTION

In one aspect, the invention relates to an osmotic intraosseous drug delivery device which comprises an artificial crown having a cavity defined therein and at least one inlet port in communication with the cavity. The device further comprises an osmotic module including an osmotic agent and a drug formulation. The osmotic module is adapted to be inserted in the cavity in order to allow fluid received in the cavity through the inlet port to activate the osmotic module. The osmotic module has an outlet port through which the drug formulation can be dispensed.

In another aspect, the invention relates to a method for intraosseous drug delivery which comprises modifying a tooth in a jawbone to provide an attachment surface for an artificial crown having a cavity defined therein and an inlet port in communication with the cavity, partially inserting an osmotic module comprising an osmotic agent and a drug formulation in a cavity in the tooth such that an outlet port of the osmotic module communicates with a root of the tooth, attaching the artificial crown to the modified tooth such that the osmotic module is sandwiched between the artificial crown and the modified tooth, receiving oral fluids in the osmotic module through the inlet port, and dispensing the drug formulation into the root.

In yet another aspect, the invention relates to a method for intraosseous drug delivery which comprises extracting a tooth from a jawbone, inserting an implant body in the jawbone in place of the tooth, inserting an osmotic module in a cavity in the implant body, wherein the osmotic module includes an osmotic agent and a drug formulation, attaching an artificial crown to the implant body such that the osmotic module is sandwiched between the artificial crown and the implant body, receiving oral fluids in the osmotic module through an inlet port in the artificial crown, and dispensing the drug formulation through an orifice provided in the implant body into the jawbone.

Other features and advantages of the invention will be apparent from the following description and the appended claims.

### BRIEF DESCRIPTION OF DRAWINGS

FIG. 1 shows an osmotic intraosseous drug delivery system including an artificial crown, a natural tooth base, and an osmotic module sandwiched between the artificial crown and the natural tooth base.

FIG. 2 shows an alternate osmotic module for the osmotic intraosseous drug delivery system depicted in FIG. 1.

FIG. 3 shows an alternate osmotic intraosseous drug delivery system including an artificial crown, an implant, and an osmotic module sandwiched between the artificial crown and the implant.

### DETAILED DESCRIPTION OF THE INVENTION

The invention will now be described in detail with reference to a few preferred embodiments, as illustrated in accompanying drawings. In the following description, numerous specific details are set forth in order to provide a thorough understanding of the invention. It will be apparent, however, to one skilled in the art that the invention may be practiced without some or all of these specific details. In other instances, well-known features and/or process steps have not been described in detail in order to not unnecessarily obscure the invention. The features and advantages of the invention may be better understood with reference to the drawings and discussions that follow.

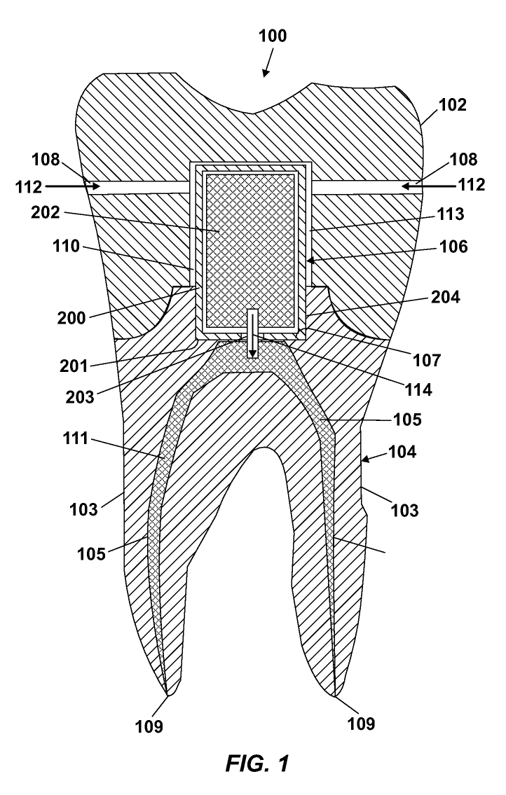

FIG. 1 is a cross-sectional view of an osmotic intraosseous drug delivery system 100 which includes an artificial tooth crown 102 mounted on a natural tooth base 104. The crown 102 is removable from the natural tooth base 104. In use, the natural tooth base 104 is embedded in a jawbone (not shown). The tooth base 104 has one or more roots 103. The term “root” generally refers to the portion of a tooth that anchors the tooth in the jawbone. There is a canal 105 in each root 103. The canal 105 can communicate with the jawbone through an opening at the tip 109 of the root 103. The canal 105 typically contains pulp/vascular tissue 111. The tooth base 104 is “natural” insofar as at least one of the roots 103 and the pulp/vascular tissue 111 therein is intact. The natural tooth base 104 has an opening or cavity 204 which is fluidly connected to the canal(s) 105. The crown 102 has a cavity 113. When the removable artificial crown 102 is attached to the natural tooth base 104 as shown, the cavities 113, 204 define a chamber 110 for receiving an osmotic module 106. The removable artificial crown 102 may be attached to the natural tooth base 104 via means such as a spring and latch, a set screw, an adhesive, or a magnetic latch. In one example, the natural tooth base 104 may be formed by removing the enamel layer of a tooth to expose the dentine layer and filing the dentine layer into a stump, which stump provides an attachment surface for the removable artificial crown 102 via a suitable means. The removable crown 102 includes inlet ports 108 that communicate with the cavity 113 in which the osmotic module 106 is partially received. The inlet ports 108 allow oral fluids 112 to enter the cavity 113 or chamber 110 in order to activate the osmotic module 106. The number of inlet ports 108 in the crown 102 is not a limitation of the invention.

The osmotic module 106 includes a rate-controlling membrane 200 and an osmotically-active drug formulation 202. The osmotically-active drug formulation 202 may be in the form of a dry compressed formulation, or solid erodible formulation. Alternatively, the osmotically-active drug formulation 202 may be a highly viscous liquid formulation. The osmotically-active drug formulation 202 may be a mixture of one or more drugs and one or more osmotic agents. The osmotic agent may be an osmotically-effective solute such as a salt or an osmotic polymer such as a hydrophilic polymer. The osmotic agent in the osmotically-active drug formulation 202 imbibes fluid into the osmotic module 106. The rate-controlling membrane 200 is formulated to regulate the amount of oral fluids 112 that pass into the osmotic module 106 in a specified time and, thus, the rate at which the osmotically-active drug formulation 202 is saturated (or eroded). The bottom 201 of the membrane 200 may include one or more openings 203 that allow the osmotically-active drug formulation 202 to flow out of the rate-controlling membrane 200 into the roots 103 of the natural tooth base 104. The rate-controlling membrane 200 may be made of a semipermeable material which would allow the oral fluids 112 to enter the osmotic module 106 and prevent the formulation within the osmotic module 106 from exiting the osmotic module except through the opening(s) 203 formed in the rate-controlling membrane 200. Where the osmotically-active drug formulation 202 is a dry compressed formulation, the oral fluids 112 imbibed into the osmotic module 106 dissolve the formulation.

In one example, the osmotic intraosseous drug delivery system 100 is installed and assembled by first grinding away a natural crown of a tooth (not shown), leaving the natural tooth base 104 of the tooth. The natural tooth base 104 is also drilled or otherwise shaped to create the opening or cavity 204 for receiving the osmotic module 106. Subsequently, the osmotic module 106 is inserted into the opening 204 of the natural tooth base 104. The cavity or opening 204 in the natural tooth base 104 is shaped to hold the osmotic module 106. The osmotic module 106 is inserted with the opening(s) 203 in the rate-controlling membrane 200 facing the base 107 of the opening or cavity 204 in the natural tooth base 104. The removable artificial crown 102 is then attached to the natural tooth base 104. The removable crown 102 appears and functions as a real tooth.

In operation, oral fluids 112 enter the chamber 110 through the inlet ports 108 in the removable crown 102. The oral fluids 112 are imbibed through the rate-controlling membrane 200 by the osmotically-active drug formulation 202 at a controlled rate. The oral fluids 112 dissolve the osmotically-active drug formulation 202 and cause the osmotically-active drug formulation 202 to expand and pass through the opening(s) 203 in the rate-controlling membrane 200 as a fluid, as indicated generally at 114. In this way, the osmotically-active drug formulation 202 acts as both an osmotic engine and a drug reservoir. The fluid 114 is absorbed into an intraosseous portal of the jawbone (not shown) in which the natural tooth base 104 is embedded. The intraosseous portal provides passage directly into the marrow of the bone (i.e., the marrow being the soft, fatty tissue that fills the bone cavity). Once most or all of the osmotically-active drug formulation 202 has been expelled from the osmotic module 106 into the intraosseous portal or after expiration of a certain time period, the entire osmotic module 106 may be replaced by a patient or a caregiver.

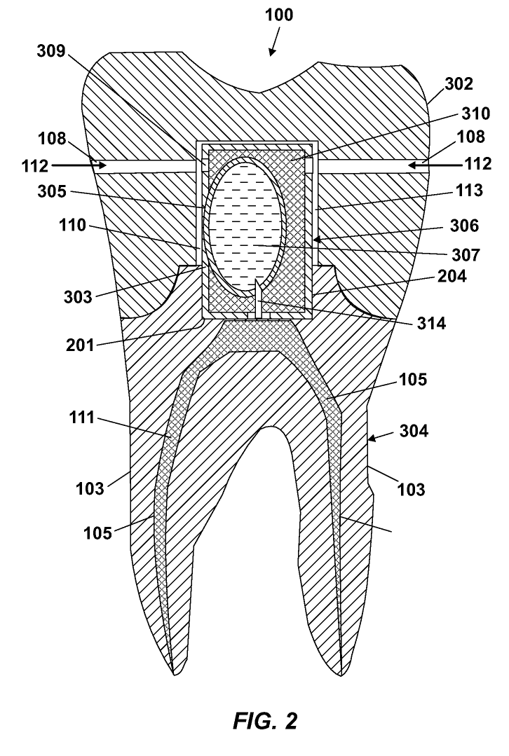

Various modifications are possible to the osmotic intraosseous drug delivery system described above. For example, in FIG. 2, the osmotic module (106 in FIG. 1) has been replaced with an osmotic module 306 including a shell 303 and a collapsible bladder 305 disposed in the shell 303. The collapsible bladder 305 stores a drug formulation 307. The shell 303 has port(s) 309 which allow oral fluids 112 received in the chamber 110 to enter the shell 303. The shell 303 may be made of implant-grade materials such as titanium. The shell 303 also contains an osmotic engine 310, which may be in the form of a fluid or may initially be solid, in addition to the collapsible bladder 305. The osmotic engine 310 includes one or more active agents which can imbibe oral fluids 1 12. In addition to osmotic agent(s), the osmotic engine 310 may include antibacterial agents to reduce the risk of infection. The osmotic engine 310 is activated by oral fluids 112 received through the ports 309 in the shell 303. The osmotic module 306 further includes an outlet needle 314 positioned at the base of the shell 303 to pierce the collapsible bladder 305 and provide a fluid passage to the natural tooth base 304. The osmotic module 306 can be installed between the removable crown 102 and the natural tooth base 104 in the same manner described for the osmotic module (106 in FIG. 1).

In operation, oral fluids 112 enter into the chamber 110 through the inlet ports 108 and into the osmotic module 306 through the ports 309. The oral fluids 312 are drawn by the osmotic engine 310 into the osmotic module 306. The osmotic engine 310 expands and, consequently, pushes the collapsible bladder 305 into contact with the outlet needle 314. The outlet needle 314 pierces the collapsible bladder 305, allowing the drug formulation 307 within the collapsible bladder 305 to flow through the outlet needle 314 into the roots 103 of the natural tooth base 104. The drug formulation is then infused into the intraosseous portal of the jawbone (not shown) in which the natural tooth base 104 is embedded.

In another example, the osmotic module 306 may not include an outlet needle 314 and both the osmotic module 306 and the collapsible bladder 305, respectively, may include an orifice for dispensing the drug formulation 307 into intraosseous portal of the jawbone (not shown) in which the natural tooth base 104 is embedded. In this example, expansion of the osmotic engine 310 continues to push the collapsible bladder 305 until the drug formulation 307 is forced out of the orifice of the collapsible bladder 305 and the orifice of the module 306 into the natural tooth base 104. When using a collapsible bladder 305, the drug formulation 307 and the osmotic engine 310 are separated, obviating the use of a piston and the like and allowing more space for the drug formulation. Once most or all of the drug formulation 307 has been expelled from the osmotic module 306 into the intraosseous portal or after expiration of a certain time period, the entire osmotic module 306 may be replaced by a patient or a caregiver.

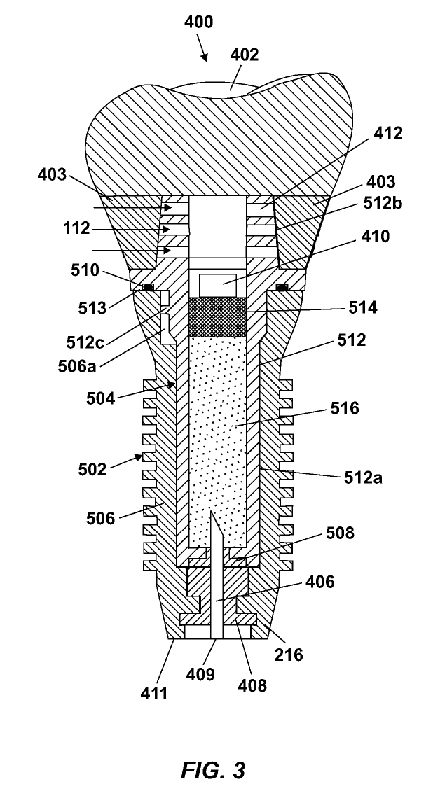

FIG. 3 shows an osmotic intraosseous drug delivery system 400 including a removable artificial crown 402, similar to artificial crown 102 in FIGS. 1 and 2, and an implant 404. In this example, the inlet port of the artificial crown 402 is provided by permeable inserts 403. It is also possible to form orifices in the artificial crown and use these orifices as inlet ports, as illustrated for the artificial crown 102 in FIGS. 1 and 2. The implant 404 is shaped for embedding or anchoring in a jawbone (not shown). The implant 404 includes an implant assembly 502 and an osmotic module or drug cartridge assembly 504. In general, the implant assembly 502 provides the infrastructure to deliver a drug stored in the drug cartridge assembly 504. The implant assembly 502 includes an implant body 506, a needle base 408, and an outlet needle 406. The needle base 408 holds the outlet needle 406. The outlet needle 406 provides a delivery orifice 409 at the base 411 of the implant body 506 and a passage for drug to enter the intraosseous portal of the jawbone. The needle base 408 may be molded to fit with the bottom of the implant body 506. The implant body 506 is a hollowed-out structure that stabilizes other components stacked inside of the implant body 506.

The drug cartridge assembly 504 includes a drug cartridge 512, a piston 410, an osmotic engine 514, a septum 508, and an O-ring seal 510. The osmotic engine 514 includes one or more osmotic agents. The osmotic agents may be osmotically-effective solutes such as salt and osmotic polymers such as hydrophilic polymers, as previously discussed. The osmotic engine 514 may be provided in the form of a tablet. The drug cartridge 512 is a replaceable reservoir for a drug formulation. The drug cartridge 512 has a cartridge body 512a and a cartridge head 512b. The cartridge body 512a has a cavity for containing the piston 410, drug formulation 516, and osmotic engine 514. When assembled, the cartridge body 512a is disposed inside the implant body 506. To prevent seepage of the drug, the septum 508 is disposed between the cartridge body 512a and the implant body 506. The outlet needle 406 pierces the septum 508, which allows fluid to be drawn from the drug cartridge 512. The cartridge head 512b rests on a lip 513 of the implant body 506. The O-ring seal 510 is used to seal the gap between the cartridge head 512b and the lip 503 of the implant body 506 such that oral fluids 112 enter the system only through the permeable inserts 403 in the artificial crown 402. The cartridge head 512b includes inlet ports 412, positioned adjacent the permeable inserts 403 of the removable crown 402. The drug cartridge 512 includes the piston 410, which drops with the level of the drug inside of the drug cartridge 512. The piston 410 is placed on top of the osmotic engine 514 but could alternately be disposed between the osmotic engine 514 and the drug formulation 516 in the cartridge body 512a. The osmotic engine 514 draws oral fluids 112 into the drug cartridge 512. The drug cartridge assembly 504 is capped by the artificial crown 402, which appears and functions as a natural tooth.

In one example, the osmotic intraosseous drug delivery system 400 is installed by first extracting a tooth from a jawbone of a patient. The implant 404 is inserted in a cavity formed in the gum of the jawbone (not shown) after extracting the tooth. In particular, the implant assembly 502 is first inserted into the cavity in the gum. After which, the drug cartridge assembly 504 is aligned and then inserted into the implant assembly 502. In one example, the drug cartridge assembly 504 and the implant assembly 502 are configured to mate with one another. For example, the drug cartridge assembly 504 may include one or more tabs 512c which mate with one or more slots 506a in the implant assembly 502. The drug cartridge assembly 504 is inserted into the implant assembly 502 until the outlet needle 406 pierces the septum 508 and the bearing surface of the drug cartridge assembly 504 contacts the bearing surface of the implant assembly 502. At this point, the O-ring seal 510 disposed between the drug cartridge assembly 504 and the implant assembly 502 is slightly deformed, thereby providing a tight seal and a low level of spring force. The compressed O-ring seal 510 between the bearing surfaces provides sufficient spring and friction forces to prevent the drug cartridge assembly 504 from rotating on its own accord. Subsequently, the drug cartridge assembly 504 is rotated, for example, in a clockwise direction, until the tabs 512c on the drug cartridge assembly 504 are locked in the slots 506a in the implant assembly 502.

Operation of the osmotic intraosseous drug delivery system 400 begins via osmotic action. Oral fluids 112 are drawn through inlet ports 412 by the osmotic engine 514 located in the drug cartridge assembly 504. The fluids force the piston 410 downwardly and the osmotic engine 514 expands, thereby pushing the drug formulation 516 stored in the drug cartridge 512 through the outlet needle 406. The outlet needle 406 provides passage for the drug to enter the intraosseous portal of the jawbone, where the drug is absorbed into the bloodstream. Once most or all of the drug formulation has been expelled from the drug cartridge 512 into the intraosseous portal or after expiration of a certain time period, the drug cartridge 512 may be replaced by a patient or a caregiver.

The drug formulations delivered by osmotic intraosseous drug delivery systems of the invention typically include one or more therapeutic agents. The therapeutic agent may be any physiologically or pharmacologically active substance, particularly those known to be delivered to the body of a human or an animal, such as medicaments, vitamins, nutrients, or the like. The therapeutic agents can be present in a wide variety of chemical and physical forms, such as solids, liquids and slurries. In addition to the one or more therapeutic agents, the drug formulation may optionally include pharmaceutically acceptable carriers and/or additional ingredients such as antioxidants, stabilizing agents, buffers, and permeation enhancers. An exemplary list of drugs and/or therapeutic agents that may be used with any of the osmotic intraosseous drug delivery system described above include but are not limited to riseperidone, hydromorphone, interferon β1a, interferon β1b, remicaid, insulin, and erythropoietin. The osmotic intraosseous drug delivery systems described above administers a drug continuously and allows refilling of the drug in a relatively non-invasive manner.

While the invention has been described with respect to a limited number of embodiments, those skilled in the art, having benefit of this disclosure, will appreciate that other embodiments can be devised which do not depart from the scope of the invention as disclosed herein. Accordingly, the scope of the invention should be limited only by the attached claims.

What is claimed is:

1. An osmotic intraosseous drug delivery device, comprising:
an artificial tooth crown having a cavity defined therein and at least one inlet port in communication with said cavity; and
an osmotic module comprising an osmotic agent and a drug formulation, said osmotic module adapted to be inserted in the cavity in order to allow fluid received in said cavity through said inlet port to activate the osmotic module, the osmotic module having an outlet port through which the drug formulation can be dispensed.

2. The osmotic intraosseous drug delivery device of claim 1, wherein the artificial crown is adapted to be removably attached to a natural tooth base.

3. The osmotic intraosseous drug delivery device of claim 1, wherein the osmotic module further comprises a rate-controlling membrane which encapsulates the osmotic agent and drug formulation.

4. The osmotic intraosseous drug delivery device of claim 3, wherein the rate-controlling membrane includes an opening through which the drug formulation can be dispensed.

5. The osmotic intraosseous drug delivery device of claim 3, wherein the osmotic agent and the drug formulation are in a solid erodible formulation.

6. The osmotic intraosseous drug delivery device of claim 1, wherein the osmotic module further comprises a collapsible bladder which encapsulates the drug formulation.

7. The osmotic intraosseous drug delivery device of claim 6, wherein the osmotic module further comprises a shell enclosing the collapsible bladder and the osmotic agent.

8. The osmotic intraosseous drug delivery device of claim 7, wherein the shell has an opening through which the drug formulation can be dispensed.

9. The osmotic intraosseous drug delivery device of claim 8, wherein a needle mounted at a base of the shell provides the opening through which the drug formulation can be dispensed, the needle being configured to pierce the collapsible bladder and receive the drug formulation from the collapsible bladder.

10. The osmotic intraosseous drug delivery device of claim 1, further comprising an implant body adapted for attachment to the artificial crown and for embedding in a jawbone.

11. The osmotic intraosseous drug delivery device of claim 10, wherein at least a portion of the osmotic module is received in a cavity defined in the implant body and the osmotic module is sandwiched between the artificial crown and the implant body.

12. The osmotic intraosseous drug delivery device of claim 11, further comprising an orifice provided at a base of the implant body through which the drug formulation can be dispensed.

13. The osmotic intraosseous drug delivery device of claim 12, wherein the orifice is provided by a needle which extends from the osmotic module to the base of the implant body.

14. The osmotic intraosseous drug delivery device of claim 11, wherein the osmotic module includes a cartridge having a cavity defined therein for containing the osmotic agent and drug formulation.

15. The osmotic intraosseous drug delivery device of claim 11, wherein the cartridge includes a port in communication with the inlet port in the artificial crown.

16. The osmotic intraosseous drug delivery device of claim 11, wherein the osmotic module further comprises a piston.

17. The osmotic intraosseous drug delivery device of claim 11, wherein the implant body sealingly engages the osmotic module such that oral fluids enter the device only through the inlet port in the artificial crown.

18. A method for intraosseous drug delivery, comprising:
modifying a tooth in a jawbone to provide an attachment surface for an artificial crown having a cavity defined therein and an inlet port in communication with the cavity;
partially inserting an osmotic module comprising an osmotic agent and a drug formulation in a cavity in the tooth such that an outlet port of the osmotic module communicates with a root of the tooth;
attaching the artificial crown to the modified tooth such that the osmotic module is sandwiched between the artificial crown and the modified tooth;
receiving oral fluids in the osmotic module through the inlet port; and
dispensing the drug formulation into a root of the tooth.

19. The method of claim 18, wherein modifying the tooth comprises removing a natural crown from the tooth and shaping the remainder of the tooth to provide the attachment surface for the artificial crown.

20. The method of claim 19, wherein modifying the tooth further comprises forming an opening in the tooth through which the outlet port of the osmotic module can communicate with the root of the tooth.

21. A method for intraosseous drug delivery, comprising:
extracting a tooth from a jawbone;
inserting an implant body in the jawbone in place of the tooth;
inserting an osmotic module in a cavity in the implant body, wherein the osmotic module includes an osmotic agent and a drug formulation;
attaching an artificial crown to the implant body such that the osmotic module is sandwiched between the artificial crown and the implant body;
receiving oral fluids in the osmotic module through an inlet port in the artificial crown;
and
dispensing the drug formulation through an orifice provided in the implant body into the jawbone.
